(19) United States
(12) Patent Application Publication
(10) Pub. No.: US 2010/0168714 A1
(43) Pub. Date: Jul. 1, 2010

(54) THERAPEUTIC AGENT DELIVERY SYSTEM

(75) Inventors: Jessica L. Burke, Bloomington, IN (US); Grant T. Hoffman, Bloomington, IN (US); Drew P. Lyons, Ellettsville, IN (US)

Correspondence Address: BRINKS HOFER GILSON & LIONE/CHICAGO/COOK, PO BOX 10395, CHICAGO, IL, 60610, US

(73) Assignee: Cook Incorpated, Bloomington, IN (US)

(21) Appl. No.: 12/529,898

(22) PCT Filed: Mar. 6, 2008
(86) PCT No.: PCT/US08/02948

§ 371 Date: Feb. 3, 2010

(30) Foreign Application Priority Data
Mar. 6, 2007 (US) 60905273

(51) Int. Cl. A61M 25/10

(52) U.S. Cl. 604/509; 604/96.01

(57) ABSTRACT

This present disclosure relates to a medical device and method capable of locally administering therapeutic agents efficiently. The medical device preferably includes one balloon catheter with conduits external to the balloon and with ports in the conduits where the conduits provide adequate sealing and sufficient penetration of the body vessel wall. Moreover, the placement of the conduits and location of the ports help ensure the optimal and sufficient administration of the therapeutic agent evenly to the entire treatment site. Other embodiments of the present disclosure relate to providing means to isolate the inflation medium and the therapeutic agent during administration to the treatment site and to introduce more than one therapeutic agent simultaneously to the treatment site.

### RELATED APPLICATIONS

This application claims priority to U.S. Provisional Patent Application No. 60/905,273, entitled “THERAPEUTIC AGENT DELIVERY SYSTEM,” filed on Mar. 6, 2007, which is incorporated herein by reference in its entirety.

### TECHNICAL FIELD

The present invention relates to medical devices configured to release a therapeutic agent. More particularly, the present invention relates to medical devices and systems commonly used with balloon catheters that locally administer therapeutic agents to a treatment site in a body vessel, as well as methods for the local administration of the therapeutic agents to a treatment site in a body vessel.

### BACKGROUND

The localized delivery of therapeutic agents within body vessels may be advantageous for treatment of a variety of medical conditions. Localized delivery may be particularly desirable for treatment of conditions that respond to administration of the therapeutic agent to a portion of a body vessel. Percutaneous delivery systems permitting administration of the therapeutic agent from a catheter placed within the body vessel permit the therapeutic agent to contact the body vessel proximate the desired treatment site. For example, vascular diseases such as atherosclerosis or peripheral vascular disease may involve stenosis of a blood vessel that may be desirably treated by administration of a therapeutic agent from a medical device within the blood vessel at or near the disease site. In general, vascular diseases may include a stenosis, i.e., the narrowing of a body vessel at stenotic lesions. Stenosis may be caused by the calcification and/or plaque (“plaque”) build-up within the body vessel. Plaque can form, for example, when cholesterol, fat and other substances form in the inner liner of the body vessel.

Angioplasty is one common treatment for stenosis. In angioplasty, balloon catheters and/or stents are used to expand the narrowed body vessel and/or treatment site. For example, Percutaneous Transluminal Coronary Angioplasty (PTCA) can widen the narrowing of a body vessel by dilation with a balloon. However, at times after PTCA an abrupt closure or more gradual closure of the body vessel occasionally follows such procedure. This phenomenon is called restenosis, which is the reoccurrence of stenosis at the treated site within the blood vessel. It is thought that restenosis may be a response to angioplasty. For instance, restenosis may result from an elastic rebound of the body vessel wall and/or by the deposition of blood platelets and fibrin along a damaged length of the newly opened body vessel near the site of an angioplasty procedure. Additionally, restenosis may result from the natural healing reaction to the injury of the body vessel wall. For example, intimal hyperplasia occurs when smooth muscle cells continuously migrate and proliferate the treatment site until the body vessel is again narrowed.

Angioplasty may include the implantation of one or more stents within a blood vessel to prevent further narrowing of the body vessel after angioplasty. Generally, the balloon catheter is positioned to predilate the stenosis in preparation of stent placement. After predilation, the stent is deployed across the treatment site once the balloon catheter is removed. One example of a device used with angioplasty to limit the slippage of the inflated device with respect to the vessel wall is U.S. Patent Application Pub. No. 2006/0085025 to Farnan et al. Here, the angioplasty balloon includes a non-deployable stent that is adapted to be secured to the balloon and to seemingly engage the vessel wall when the balloon is in the expanded state. However, restenosis may still occur over the length of the stent and/or past the ends of the stent where the inward forces of the stenosis are unopposed. Besides restenosis, tumor formation and thrombosis, the formation of a fibrinous clot in a blood vessel, are other common drawbacks associated with stent placement during angioplasty.

As a result, procedures have been developed using catheters to deliver therapeutic agents to the treatment site within a body vessel to mitigate or eliminate conditions such as restenosis, tumor formation and/or thrombosis. Some catheters, such as U.S. Pat. No. 4,423,725 to Baran, comprise of a plurality of balloons where two expanded balloons each located on the outermost extremes of the treatment site isolate the treatment site in preparation for the administration of the therapeutic agents from the catheter in the region between the balloons. After inflation of the two balloons, the therapeutic agent may be locally introduced from apertures in the catheter. However, the inflation of the multiple catheter balloons may require undesirably extended inflation time and/or dwell time of the catheter.

Some medical devices comprise catheters with a single balloon and a plurality of perforations or ports. For example, U.S. Pat. No. 5,112,305 to Barath, describes such a device, and related method of use, relating to a double lumen catheter having tubular extensions in communication with a drug-delivery and inflation lumen. The tubular extensions protrude at various angles from the outermost surface of the balloon. Upon inflation of the balloon in a body vessel, the tubular extensions penetrate the body vessel wall, and a therapeutic agent then is propelled through the tubular extensions into the wall of the body vessel. Although the tubular extensions may provide adequate sealing to localize the administration of the therapeutic agent within the body vessel wall, the projections may not ensure even administration of the therapeutic agent along the entire treatment site, and excessive amounts of the therapeutic agent may be needed to ensure adequate treatment. Furthermore, the inflation medium and the therapeutic agent are mixed together before being administered to the treatment site, preventing the simultaneous administration of different therapeutic agents from different tubular extensions.

Another example of a catheter adapted for the localized administration of a therapeutic agent is U.S. Pat. No. 5,232,444 to Just et al., which describes a balloon catheter with a plurality of pore-like apertures in the balloon. The therapeutic agent is disposed inside the balloon with the dilating medium containing the therapeutic agent. One embodiment has a plurality of compartments within the balloon, which are sealed from one another with neighboring compartments accommodating different therapeutic agents. Although the porous balloon catheter can accommodate the simultaneous introduction of more than one therapeutic agent, the therapeutic agent(s) are administered through the pores of the balloon surface that are not embedded within the walls of the body vessel, permitting the therapeutic agent to be carried through the body vessel during the delivery process. This may require excessive amounts of therapeutic agent to ensure administration of adequate amount of the therapeutic agent to the wall of the body vessel.

Angioplasty may also include an atherotomy procedure, or cutting balloon angioplasty. Cutting balloons conventionally consist of a plurality of cutting edges, or atherotomes, mounted longitudinally along the surface of an inflatable balloon. With dilation of the balloon at a treatment site within a body vessel, the cutting edges can score the plaque and can press fatty matter into the vessel wall. The dilation pressure of the cutting balloons is generally less than the dilation pressure of balloons used in PCTA. Also, less force may be applied to the vessel wall with less abruptness. Therapeutic agents can also be delivered to the treatment site after cutting balloon angioplasty. Once introduced, therapeutic agents can be locally introduced from the apertures of the infusion catheter. The therapeutic agents treat restenosis after the cutting balloon is removed. Nevertheless, the use of cutting balloons can damage and traumatize the body vessel wall to a degree of leading to restenosis.

One example of a cutting balloon which incorporates the delivery of a therapeutic agent is U.S. Patent Application Pub. No. 2006/0259005 to Konstantino et al. Here, the methods and systems for providing a drug to a luminal site includes angioplasty balloon with scoring elements adapted to deliver a drug. The scoring elements can include a well or a horizontal through hole where the drug is applied to the elements before being introduced to the body lumen. After positioning the scoring elements at the luminal site, the scoring elements can engage the wall of the body lumen, which typically involves the radial expansion of an expandable shaft or balloon. Once the scoring elements are engaged into the wall of the body lumen, the drug can then be released. However, these systems and methods require a scoring of the body vessel wall prior to releasing the drug to locations in or beneath the intimal layer of the body vessel wall. The scoring members penetrate the wall of the body vessel to contain the released drug within the incision formed by the scoring process. While the scoring of the vessel wall permits delivery of the drug to intimal or subintimal layers surrounding the blood vessel, the scoring process also damages the vessel wall. This damage to the vessel wall may, in turn, lead to additional complications, such as thrombus formation or inflammation of the scoring site. What is needed are improved systems and methods for delivering a therapeutic agent to a body vessel using a catheter-based delivery system without the need to score the body vessel.

There is a need for a medical device, and method of related use, capable of locally administering a therapeutic agent efficiently to a treatment site within a body vessel, for example to mitigate the occurrence of restenosis, tumor formation and thrombosis during or after angioplasty procedures. In particular, there is a need for a medical device adapted to disperse a therapeutically effective dose of a therapeutic agent to a localized area of a body vessel wall without substantial loss of the therapeutic agent from the treatment area. For example, medical devices adapted to release a therapeutic agent into a sealed area of the body vessel wall may be desirable for such an application. In addition, there is also a need for medical devices adapted to simultaneously administer desired amounts of multiple therapeutic agents to two separate treatment site areas within a single body vessel. When administering therapeutic agents into a vessel, it is more efficient to deliver the drug directly to the body vessel wall as opposed to infusing the lumen with a larger amount of the drug with the hope that enough of the therapeutic agent interacts with the body vessel wall before being transported further down the vessel by blood flow. Finally, there is also a need to provide catheter-based means for delivering one or more therapeutic agents a body vessel wall and maintaining the delivered therapeutic agent in intimate contact with the body vessel wall without cutting the body vessel wall.

### BRIEF SUMMARY

This present disclosure relates to medical devices and methods for locally administering therapeutic agents within a body vessel. The medical devices preferably include one balloon catheter with one or more conduits external to the balloon. The conduits preferably include drug delivery ports and may be configured to provide adequate sealing between the port and the body vessel wall, as well as sufficient penetration of the port within the body vessel wall. Moreover, the position and configuration of the conduits and location of the ports may be selected to provide local administration of the therapeutic agent evenly to an entire treatment site within the body vessel. The conduits may be formed from a material having a rigidity sufficient to permit the body vessel to enclose the conduit upon expansion of the balloon in a manner to force the conduit into the wall of the body vessel without cutting the wall of the body vessel. Other embodiments relate to providing more than one therapeutic agent simultaneously to the treatment site.

According to a first embodiment, a therapeutic agent delivery system comprises a catheter shaft and a therapeutic agent delivery conduit. The catheter shaft has an expandable portion that is inflatable from a deflated configuration to an inflated configuration. The catheter shaft may extend along a longitudinal axis from a proximal end to a distal end and may include an inflation lumen in communication with the expandable portion. The therapeutic agent delivery conduit may include a therapeutic agent delivery lumen and also include a therapeutic agent delivery port in communication with the therapeutic agent delivery lumen. The therapeutic agent delivery conduit is preferably positioned external to the expandable portion of the catheter shaft and may contact at least a portion of an external surface of the expandable portion while the expandable portion is in the inflated configuration. The therapeutic agent delivery conduit desirably moves independently of the expandable portion while the expandable portion is in the deflated configuration.

In a first aspect of the first embodiment, the therapeutic agent delivery system can comprise a plurality of therapeutic agent delivery ports. The plurality of ports are preferably located along one or more therapeutic agent conduits and can face radially away from the portion of the external surface of the expandable portion that contacts the therapeutic agent delivery conduit. Preferably, the plurality of therapeutic agent delivery ports are disposed longitudinally along the therapeutic agent delivery conduit(s) in a substantially straight line. Furthermore, the plurality of therapeutic agent delivery ports can include a first therapeutic agent delivery port located distally to a second therapeutic agent delivery port, where the first therapeutic agent delivery port has a larger cross-sectional area than the second therapeutic agent delivery port.

In a second aspect of the first embodiment, the therapeutic agent delivery system can further comprise a second therapeutic agent delivery conduit. The second therapeutic agent delivery conduit includes a second therapeutic agent delivery lumen and also includes a second therapeutic agent delivery port in communication with the therapeutic agent delivery lumen. The second therapeutic agent delivery conduit is positioned external to the expandable portion of the catheter shaft and contacts at least a portion of an external surface of the expandable portion while the expandable portion is in the inflated configuration. The second therapeutic agent delivery conduit also moves independently of the expandable portion while the expandable portion is in the deflated configuration.

In a third aspect of the first embodiment, the therapeutic agent delivery system can further comprise a distal tip. A distal end of the first therapeutic agent delivery conduit and a distal end of the second therapeutic agent delivery conduit are joined to form the distal tip. The distal tip is positioned distal to the expandable portion of the catheter shaft. Furthermore, the distal tip includes an annular opening adapted for receiving a guide wire and moves independently of the expandable portion in the deflated configuration. Additionally, the first portion of the external surface of the expandable portion and the second portion of the external surface of the expandable portion can be spaced such that a circumferential distance between each portion, measured perpendicular to the longitudinal axis, is substantially equal.

In a fourth aspect of the first embodiment, a therapeutic agent delivery device comprises a first therapeutic agent delivery conduit, a second therapeutic agent delivery conduit, a distal tip, and a proximal base. The distal tip preferably joins the first and second therapeutic agent delivery conduits. The distal tip may also include an annular opening aligned along a longitudinal axis. The proximal base is separated proximally from the distal tip by a longitudinal distance. The proximal base joins the first and second therapeutic agent delivery conduits and is preferably adapted to receiving a catheter shaft. The first therapeutic agent delivery conduit and the second therapeutic agent delivery conduit are desirably separated from each other and are preferably adapted to move independently from each other between the distal tip and the proximal base. The first therapeutic agent delivery conduit may include a first therapeutic agent delivery lumen and also a therapeutic agent delivery port in communication with the first therapeutic agent delivery lumen. The second therapeutic agent delivery conduit may also include a second therapeutic agent delivery lumen and a second therapeutic agent delivery port in communication with the second therapeutic agent delivery conduit. Each therapeutic agent delivery conduit is preferably positioned at a radial distance from the longitudinal axis in a low-profile configuration. In the low-profile configuration, the longitudinal distance between the distal tip and the proximal base is at or near a maximum longitudinal separation distance. Each therapeutic agent delivery conduit is preferably configured to bend resiliently from a low-profile configuration to an expanded configuration upon longitudinal translation of the distal tip toward the proximal base along the longitudinal axis. Preferably, the radial distance of at least a portion of each therapeutic agent delivery conduit from the longitudinal axis increases when each therapeutic agent delivery conduit is moved from the low-profile configuration to the expanded configuration.

In a second embodiment, methods of treatment are provided that relate to delivering a therapeutic agent(s) to an interior wall of a body vessel at or near a treatment site using a medical device described according to the first embodiment. In one aspect, methods of delivering a therapeutic agent delivery system are provided that include administration of a therapeutic agent from one or more therapeutic agent delivery ports in a therapeutic agent delivery conduit. The conduit may be attached to a catheter shaft having an expandable portion that is inflatable from a deflated configuration to an inflated configuration. The therapeutic agent delivery system may be inserted into a body vessel. A portion of the therapeutic agent delivery system may be translated within the body vessel until a therapeutic agent delivery conduit with the therapeutic agent delivery port is positioned proximate the treatment site within the body vessel. The expandable portion of the catheter shaft may be inflated within the body vessel until at least the portion of the external surface of the expandable portion contacts the therapeutic agent delivery conduit. The pressure of the expandable portion of the catheter shaft may be increased until the therapeutic agent delivery conduit is pressed into the wall of the body vessel. A therapeutic agent may be injected from the therapeutic agent delivery lumen through the therapeutic agent delivery port to the wall of the body vessel proximate the treatment site. A portion of the body vessel may enclose one or more ports in the conduit, permitting the injected therapeutic agent to remain in an interstitial space between the body vessel wall and the conduit port while the therapeutic agent diffuses into the body vessel wall tissue. In this manner, the therapeutic agent may be absorbed by the body vessel without scoring or cutting the wall of the body vessel. After treatment, the expandable portion of the catheter shaft may be deflated and the therapeutic agent delivery system removed from the body vessel. The therapeutic agent absorbed by the wall of the body vessel may diffuse slowly through multiple layers of the body vessel tissue after treatment, permitting the gradual administration of the therapeutic agent throughout the body vessel after removal of the conduit from the body vessel.

### BRIEF DESCRIPTION OF THE DRAWINGS

The following detailed description of certain exemplary embodiments can be understood when with reference to the following drawings, where like structure is indicated with like reference numerals and in which:

FIG. 1 is a perspective view of a first therapeutic agent delivery system in the deflated configuration.

FIG. 2 is a cross sectional view taken along line 2-2 of the first therapeutic agent delivery system in FIG. 1.

FIG. 3 is a cross sectional view taken along line 3-3 of the first therapeutic agent delivery system in FIG. 1.

FIG. 4a is a cross sectional view taken along line 4-4 of the first therapeutic agent delivery system in FIG. 1 showing the inflation lumen coaxial with the guide wire and the therapeutic agent delivery lumen.

FIG. 4b is a cross sectional view taken along line 4-4 of the first therapeutic agent delivery system in FIG. 1 showing the inflation lumen and the therapeutic agent delivery lumen contained within the catheter shaft.

FIG. 5 is a perspective view of a first therapeutic agent delivery system in the inflated configuration.

FIG. 6 is a cross sectional view taken along line 6-6 of the first therapeutic agent delivery system in FIG. 5.

FIG. 7 is a cross sectional view taken along line 7-7 of the first therapeutic agent delivery system in FIG. 5.

FIG. 8 is an enlarged proximal portion of the first therapeutic agent delivery system of FIG. 1 and FIG. 5.

FIG. 9 is an enlarged proximal portion of the first therapeutic agent delivery system of FIG. 1 and FIG. 5.

FIG. 10 is an enlarged cut-away view of the distal portion of the first therapeutic agent delivery system of FIG. 1 and FIG. 5.

FIG. 11 is a perspective view of a second therapeutic agent delivery device in the low-profile configuration.

FIG. 12 is a perspective view of the second therapeutic agent delivery device in the expanded configuration.

FIG. 13 is a cross sectional view taken along line 13-13 of the second therapeutic agent delivery device in FIG. 11.

FIG. 14 is a cross sectional view taken along line 14-14 of the second therapeutic agent delivery device in FIG. 12.

FIG. 15 is a side elevation view, partially in section, of the second therapeutic agent delivery system in the inflated configuration being applied within the body vessel.

FIG. 16 is a cross sectional view taken along line 16-16 of the second therapeutic agent delivery device in FIG. 15.

### DETAILED DESCRIPTION

As used herein, the term “implantable” refers to an ability of a medical device to be positioned at a location within a body, such as within a body vessel. Furthermore, the terms “implantation” and “implanted” refer to the positioning of a medical device at a location within a body, such as within a body vessel.

The term “biocompatible” refers to a material that is substantially non-toxic in the in vivo environment of its intended use, and that is not substantially rejected by the patient's physiological system (i.e., is non-antigenic). This can be gauged by the ability of a material to pass the biocompatibility tests set forth in International Standards Organization (ISO) Standard No. 10993 and/or the U.S. Pharmacopeia (USP) 23 and/or the U.S. Food and Drug Administration (FDA) blue book memorandum No. G95-1, entitled “Use of International Standard ISO-10993, Biological Evaluation of Medical Devices Part-1: Evaluation and Testing.” Typically, these tests measure a material's toxicity, infectivity, pyrogenicity, irritation potential, reactivity, hemolytic activity, carcinogenicity and/or immunogenicity. A biocompatible structure or material, when introduced into a majority of patients, will not cause an undesirably adverse, long-lived or escalating biological reaction or response, and is distinguished from a mild, transient inflammation which typically accompanies surgery or implantation of foreign objects into a living organism.

As used herein, the term “body vessel” means any body passage lumen that conducts fluid, including but not limited to blood vessels, esophageal, intestinal, billiary, urethral and ureteral passages.

The medical devices of the embodiments described herein may be oriented in any suitable absolute orientation with respect to a body vessel. The recitation of a “first” direction is provided as an example. Any suitable orientation or direction may correspond to a “first” direction. For example, the first direction can be a radial direction in some embodiments.

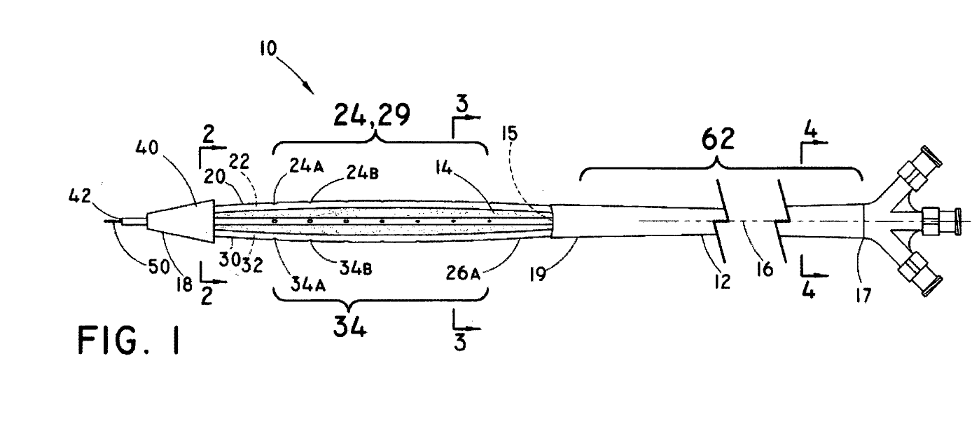

FIG. 1 and FIG. 5 show an exemplary embodiment of a first therapeutic agent delivery system 10 comprising a catheter shaft 12 extending along a longitudinal axis 16 and a therapeutic agent delivery conduit 20. The catheter shaft 12 has an expandable portion 14 which is inflatable from a deflated configuration (as shown in FIG. 1) to an inflated configuration (as shown in FIG. 5). The expandable portion 14 is preferably a balloon or other similar type structure used in the art for angioplasty treatment of a stenosis. The catheter shaft 12 includes an inflation lumen 15 in communication with the expandable portion 14. The inflation of the expandable portion 14 is accomplished by any suitable means known in the art, e.g., by introducing an inflation fluid (e.g., air, saline, etc.) through the inflation lumen 15 into the expandable portion 14. The catheter shaft 12 extends along the longitudinal axis 16 from a proximal end 17 to a distal end 18.

The expandable portion 14 comprises any suitably non-elastic material such as linear low density polyethylene, polyethyleneterephthalate (PET), polyurethanes, irradiated polyethylene, ionomers, copolyesters, rubbers, polyamides including nylons, polyester, or any medical grade polymers suitable for use in forming catheter balloons. Preferably, the geometry, material and configuration of the expandable portion 14 is selected to withstand an internal inflation fluid pressure of about 5 atmospheres and, preferably, about 10 atmospheres without any leakage or rupture. The thickness of the expandable portion 14 and the catheter shaft 12 should be selected to provide an expandable portion 14 that will exert sufficient force against the luminal wall without rupturing, while providing sufficient radial force to direct the conduit 20 into the body vessel wall. The expandable portion 14 and the catheter shaft 12 may have any suitable dimension, but is preferably shaped and configured for the intended use in a body vessel. The catheter shaft 12 preferably includes a lumen configured to house a guide wire 50. For example, the guide wire 50 lumen of the catheter shaft 12 may have an inside diameter of about approximately 0.5 mm. The catheter shaft 12 may have any suitable length for an intended use, such as approximately 110-180 cm. The catheter shaft 12 may optionally be configured as a rapid exchange catheter, such as the catheter devices described in U.S. Pat. Nos. 5,690,642 and 5,814,061. In a rapid exchange configuration, the proximal terminus of the guide wire 50 lumen may be positioned distal to the proximal end 17 of the catheter shaft 12. For example, the guide wire 50 lumen may extend from the distal end 18 of the catheter shaft 12 to a rapid exchange access port positioned at least 5, 10 or 15 cm distal to the distal end 17 of the catheter shaft 12. The outside diameter of the catheter shaft 12 is typically approximately 1-1.5 mm. When configured for use in a peripheral blood vessel, the inflated diameter of the expandable portion 14 may be selected based on the diameter of a body vessel. Typically, the inflated diameter of the expandable portion 14 is at least about 1-5% greater than the diameter of the body vessel at a treatment site. For example, the expandable portion 14 may be placed at a treatment site that is a stenosis in a body vessel, and expanded to an outer diameter of about 1.5 mm to about 8 mm. For a treatment site intended for coronary vascular applications, the outer diameter of the expandable portion 14 preferably expands to an inflated diameter in the range of about 1.5 mm to about 4 mm. When configured for use in bile ducts, the expanded diameter of the expandable portion 14 may be about 5-15 mm with a length of approximately 15-60 mm, the outer diameter of the catheter shaft 12 may be up to about 3.5 mm.

Referring again to FIG. 1 and FIG. 5, the first therapeutic agent delivery system 10 further comprises a therapeutic agent delivery conduit 20, which includes a therapeutic agent delivery lumen 22 in communication with a therapeutic agent delivery port 24. FIG. 7 is a cross sectional view taken along line 7-7 of the therapeutic agent delivery system as shown in FIG. 5. With reference to FIG. 7, the expandable portion 14 is inflated and the therapeutic agent delivery conduit 20 is positioned external to the expandable portion 14 and is in contact with at least a portion 23 of an external surface 13 of the expandable portion 14 while the expandable portion 14 is in the inflated configuration. Portions of the inflated expandable portion 14 may radially expand between the conduits 20, 30 and may urge the conduits 20, 30 radially outward from the longitudinal axis 16. The therapeutic agent delivery conduit 20 can be disposed substantially parallel to the longitudinal axis 16 while the expandable portion 14 is in the deflated configuration, as shown in FIG. 1. Referring to FIG. 1, the therapeutic agent delivery conduit 20 is moveable independent of the expandable portion 14 while the expandable portion 14 is in the deflated configuration. FIG. 3 is a cross sectional view taken along line 3-3 of the therapeutic agent delivery system in FIG. 1, with the expandable portion 14 deflated. FIG. 3 shows the conduit 20 being spaced apart from the expandable portion 14 to permit independent movement of the therapeutic agent delivery conduit 20 from the expandable portion 14. Optionally, as shown in FIG. 3, the therapeutic agent delivery conduit 20 may be radially spaced apart from the expandable portion 14 of the catheter shaft 12.

In one aspect of the first embodiment, the therapeutic agent delivery system 10 preferably includes multiple therapeutic agent delivery conduits 20, 30. For example in the first therapeutic agent delivery system 10 shown in FIG. 1 and FIG. 5, the system 10 contains a second therapeutic agent delivery conduit 30 along with two other conduits (one shown and one conduit being positioned behind the expandable portion 14 and not shown in FIG. 1 or FIG. 5). The second therapeutic agent delivery conduit 30 is preferably substantially similar or identical to the first therapeutic agent delivery conduit 20 except with respect to its position and orientation relative to the expandable portion 14 of the catheter shaft 12. Similar to the first therapeutic agent delivery conduit 20, the second therapeutic agent delivery conduit 30 preferably includes a second therapeutic agent delivery lumen 32 and a second therapeutic agent delivery port 34. The second therapeutic agent delivery port 34 is in communication with the second therapeutic agent delivery lumen 32. The second therapeutic agent delivery conduit 30 may be disposed substantially parallel to the longitudinal axis 16 while the expandable portion 14 is in the deflated configuration. As shown in FIG. 7, the second therapeutic agent delivery conduit 30 is positioned external to the expandable portion 14 and is in contact with at least a second portion 33 of an external surface 13 of the expandable portion 14 while the expandable portion 14 is in the inflated configuration. The second therapeutic agent delivery conduit 30 may be spaced apart from and moveable independent of the expandable portion 14 while the expandable portion 14 is in the deflated configuration. FIG. 3 also shows the second conduit 30 being detached from, and spaced apart from, the expandable portion 14 to permit independent movement of the therapeutic agent delivery conduit 20 from the expandable portion 14.

The therapeutic agent delivery conduits may be oriented in any suitable direction with respect to the longitudinal axis 16. In the first therapeutic agent delivery system 10, the therapeutic agent delivery conduits 20, 30 are oriented substantially parallel to the longitudinal axis 16. Alternatively, the therapeutic agent delivery conduits 20, 30 can also be arranged in a spiral pattern around the expandable portion 14 of the catheter shaft 12. The conduit is preferably a separate tube from the expandable portion 14.

The therapeutic agent delivery conduits 20, 30 may be made of any material. Preferably, the material is selected to be more rigid than the material of the expandable portion 14. Preferred materials are sufficiently rigid to maintain the patency of the lumens 22, 32 within the conduits 20, 30, as well as the drug delivery ports 24, 34, upon compression of the conduits 20, 30 between the expanded expandable portion 14 and the wall of a body vessel. The materials may be selected to have a rigidity that permits the conduits 20, 30 to maintain a substantially constant cross sectional shape and volume within the drug delivery lumen while passing a fluid comprising a therapeutic agent therethrough at a desired rate and pressure. Preferred materials are thermoformable medical-grade polymers such as polyethylene or polyurethane polymers or co-polymers. Optionally, the therapeutic agent delivery conduits may include a radiopaque material permitting identification of the location and orientation of the conduit(s) within a body vessel by a suitable medical imaging technique such as fluoroscopy.

In the therapeutic agent delivery system 10 of FIG. 1 and FIG. 5, the conduit 20 may have a plurality of therapeutic agent delivery ports 24, as well as the conduit 30 may have a plurality of therapeutic agent delivery ports 34. Other embodiments can have one therapeutic agent delivery port 24. Ports 24, 34 are desirably arranged and oriented away from the expandable portion 14. In use, inflation of the expanded portion 14 of the catheter shaft 12 presses the conduit 20 into the body vessel wall. A therapeutic agent may be injected into the therapeutic agent delivery lumen 22 of the conduit 20, 30 and released at the treatment site within the portion of the body vessel radially distended away from the longitudinal axis 16 by the conduits 20, 30. Upon inflation of the expandable portion 14, the ports 24, 34 can face away from the portion 23 of the external surface 13 of the expandable portion 14 that is in contact with the therapeutic agent delivery conduit 20, 30 as shown in FIG. 7. Furthermore, the ports 24, 34 can be disposed longitudinally along the conduit 20, 30, for example in a substantially straight line as shown in FIG. 1 and FIG. 5. The ports 24 can be located along on only a portion 29 of the therapeutic agent delivery conduit 20. A portion 29 comprising the ports 24 may be positioned external to the expandable portion 14 of the catheter shaft 12 and can be disposed substantially parallel to the longitudinal axis 16 while the expandable portion 14 is in the deflated configuration (FIG. 1).

In another aspect of the first embodiment of the therapeutic agent delivery system 10 may include ports 24 having different cross-sectional areas. Preferably, the cross-sectional area of the ports 24 increase moving in the distal direction along the longitudinal axis 16 to compensate for the fluid pressure losses associated with the walls of the conduit 20 and the ports 24 and to provide a more evenly distribution of the release of the therapeutic agent at the treatment site. As shown in FIG. 1, a first therapeutic agent delivery port 24a located distally to a second therapeutic agent delivery port 24b. The first therapeutic agent delivery port 24a may have a larger cross-sectional area than the second therapeutic agent delivery port 24b. Similarly, the plurality of ports 34 may include a distal therapeutic agent delivery port 34a located distally to proximal therapeutic agent delivery port 34b. The distal therapeutic agent delivery port 34a may have a larger cross-sectional area than the proximal therapeutic agent deliver port 34b. The plurality of ports 34 may be similarly sized and situated as the plurality of ports 24, or may be differently sized or situated. The therapeutic agent delivery ports 24, 34 can be made in any traditional manner, preferably with either sideport machine or drill. Further with respect to accommodating fluid pressure losses and providing even distribution, the lumens 22, 32 may also have portions of different cross-sectional areas. For example, the cross-sectional area of the lumens 22, 32 may decrease or taper distally along the lumen.

The expandable portion 14 may have a longitudinally curved external surface 27 while in the inflated configuration, as shown in FIG. 5. Inflation of the expandable portion 14 from the deflated configuration to the inflated configuration may bend the therapeutic agent delivery conduit 20 from a substantially straight configuration 26a (as shown in FIG. 1) to a substantially arcuate configuration 26b (as shown in FIG. 5) along the longitudinally curved external surface 27 of the expandable portion 14 in the inflated configuration.

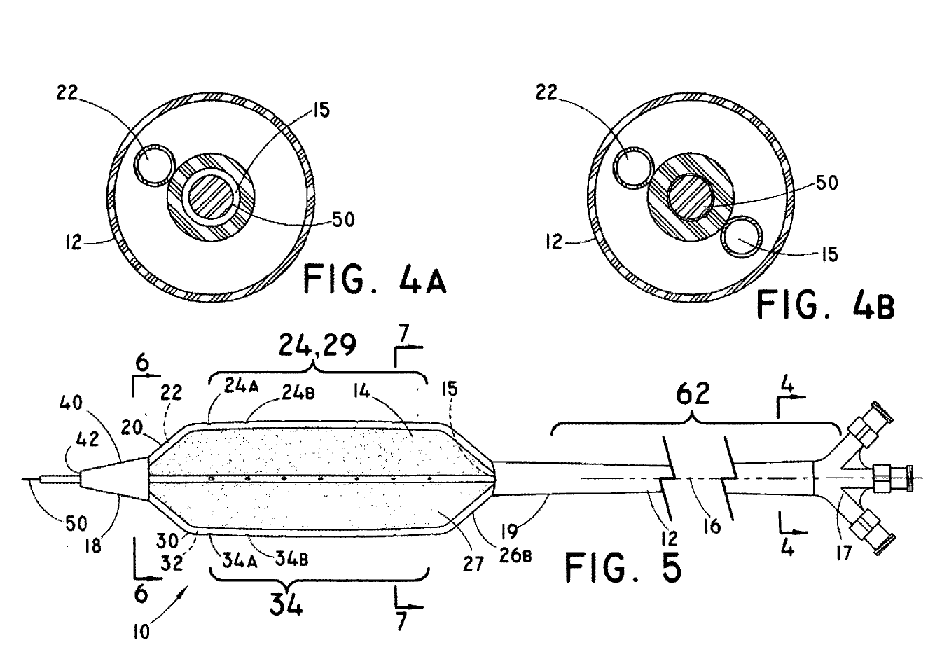

Preferably, the catheter shaft 12 houses one or more therapeutic agent delivery lumens 22 in communication one or more therapeutic agent delivery conduits 20 and a separate inflation lumen 15 in communication with the expandable portion 14. At least a portion of the catheter shaft 12 may contain a third lumen adapted to receive a guide wire 50 or stiffening member. The catheter shaft 12 may alternatively be configured as a rapid exchange catheter (not shown). FIG. 4a and FIG. 4b are cross sectional views taken along line 4-4 of the therapeutic agent delivery system in FIG. 1 showing two different examples of suitable catheter shaft 12 configurations. The proximal portion 62 of the catheter shaft 12 shown in FIG. 1 and FIG. 5 extends from a proximal end 19 of the expandable portion 14 to the proximal end 17 of the catheter shaft 12. Therein, the therapeutic agent delivery lumen 22 and the inflation lumen 15 may be coaxially oriented along the longitudinal axis 16 from the proximal end 17 of the catheter shaft 12 to the proximal end 19 of the expandable portion 14 as shown in FIG. 4a. Alternatively, FIG. 4b is a cross sectional view taken along line 4-4 of the therapeutic agent delivery system in FIG. 1 showing the inflation lumen 15 and the therapeutic agent delivery lumen 22 arranged side-by-side with the lumen housing the guide wire 50 within the catheter shaft 12.

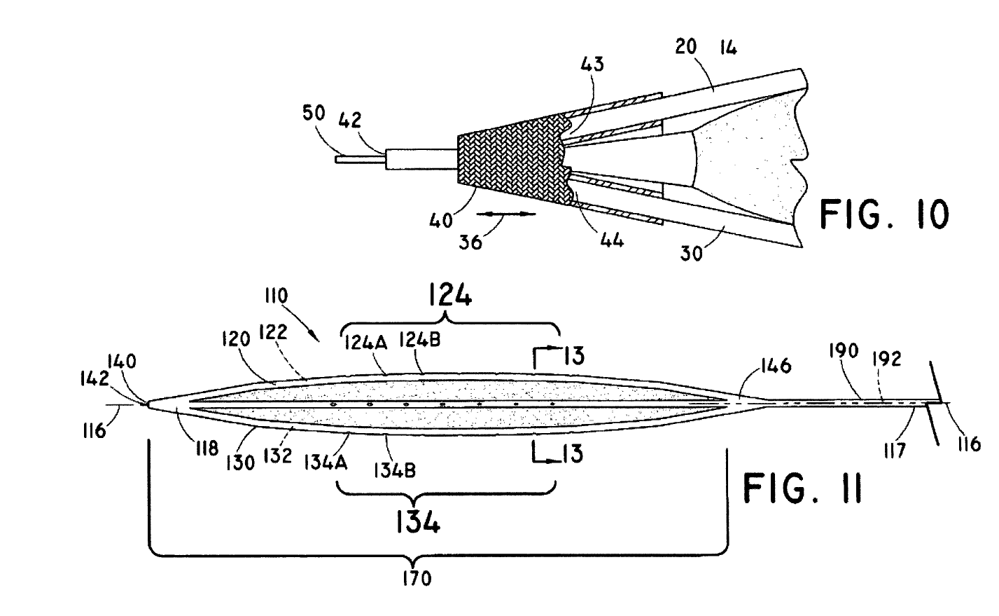

FIG. 8 and FIG. 9 are detailed cut-away views of two different proximal end 19 configurations of the expandable portion 14 shown in FIG. 1 and FIG. 5. In FIG. 8, a first configuration of the proximal portion 62 of the catheter shaft 12 can include a portion of a single therapeutic agent delivery lumen 22 proximal to, and in communication with, the therapeutic agent delivery conduit 20. Alternatively, in FIG. 9, a second configuration of the proximal portion 62 of the catheter shaft 12 can include more than one therapeutic agent delivery lumen 22, 32 proximal to, and in communication with, each respective therapeutic agent delivery conduit 20, 30. The second configuration (FIG. 9) permits therapeutic agents to be delivered to different conduits from separate therapeutic agent delivery lumens 22, 32 simultaneously, whereas a single first configuration (FIG. 8) delivers a single stream of the therapeutic agent from one therapeutic agent delivery lumen 22 to multiple conduits 20. In both of the configurations shown in FIG. 8 and FIG. 9, the inflation medium and the therapeutic agent are isolated from each other because of the separate walls of the lumen 15 of the catheter shaft and the lumen 22 of the therapeutic agent delivery conduit 20. Also, because more than one therapeutic agent delivery conduit 20, 30 are present in FIG. 9, more than one therapeutic agent can be administered simultaneously to the treatment site. As a result, multiple therapeutic agents can be delivered to the treatment site.

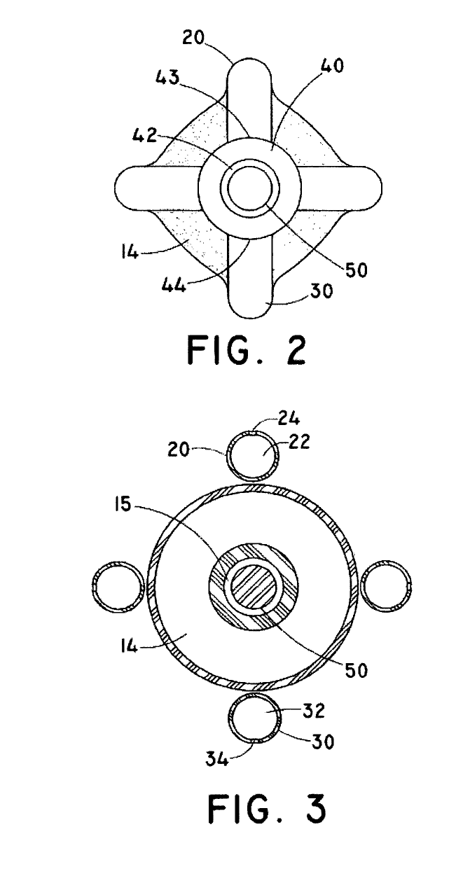

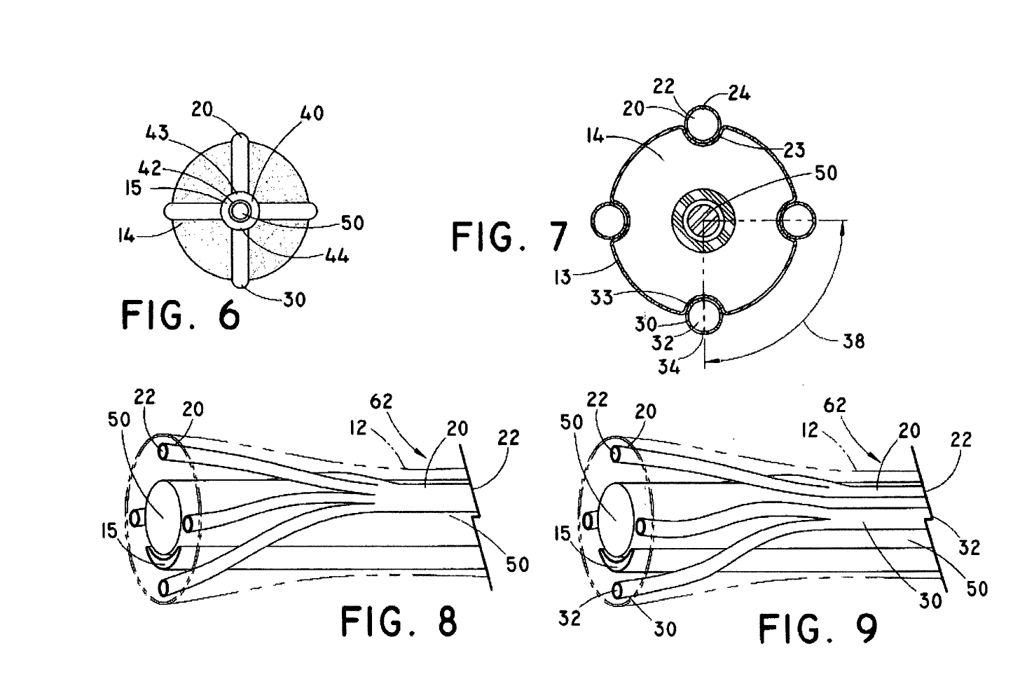

FIG. 10 is a cut-away view of a distal tip 40 positioned at the distal end of the medical device shown in FIGS. 1-9. FIG. 2 is a cross sectional view of a distal tip 40 taken along line 2-2 of the therapeutic agent delivery system in FIG. 1 and FIG. 6 is a cross sectional view of a distal tip 40 taken along line 6-6 of the therapeutic agent delivery system in FIG. 5. FIG. 2, FIG. 6 and FIG. 10 further illustrate the distal tip 40 formed where a distal end 43 of the first therapeutic agent delivery conduit 20 and a distal end 44 of the second therapeutic agent delivery conduit 30 are joined. The distal tip 40 is positioned distal to the expandable portion 14 of the catheter shaft 12. The distal tip 40 includes an annular opening 42 adapted for receiving a guide wire 50.

The distal tip 40 is preferably unattached to the expandable portion 14. The distal tip 40 is also preferably moveable independent of the expandable portion 14 in the deflated configuration. For example, the distal tip 40 may be longitudinally moveable with respect to the expandable portion 14 and the catheter shaft 12. The distal tip 40 is preferably joined to one or more of the conduits 20, 30. Most preferably, all of the conduits 20, 30 are joined together at the distal tip 40, without being attached to the expandable portion 14. Upon inflation of the expanded portion 14 of the catheter shaft 12 and subsequent radial expansion of the conduits 20, 30 away from the longitudinal axis 16, the distal tip 40 may translate longitudinally toward the proximal end 19, as represented by arrows 36. As shown in FIG. 10, the distal tip 40 is preferably adapted to translate longitudinally along the guide wire 50, substantially parallel to the longitudinal axis 16 of the catheter shaft 12. Alternatively, in alternative embodiments, the distal tip 40 may be attached to the distal end of the expandable portion 14, and the conduits 20, 30 may have sufficient elasticity to allow for expansion of the conduits 20, 30 with respect to each other without cracking or breaking the distal tip 40.

The first therapeutic agent delivery system 10 may include any suitable number of conduits, including one, two, three, four, five, six, seven, eight or more conduits. Preferably, the conduits are substantially equally spaced with respect to one another around the circumference of the inflated expandable portion 14. In other words, the radial angle from the longitudinal axis 16 between the centers of adjacent conduits is preferably 2 π/n radians, where n is an integer equal to the total number of conduits (e.g., n=1, 2, 3, 4, 5, 6, 7, 8 or more). As shown in FIG. 7, a first portion 23 and a second portion 33 of the external surface 13 of the expandable portion 14 can be spaced such that a circumferential distance 38 between each portion, measured perpendicular to the longitudinal axis, is substantially equal. Equal spacing will likely produce better local distribution of the therapeutic agent along the entire treatment site. However, the circumstantial distance 38 need not be substantially equal. Alternatively, one or more of the conduits may also be oriented helically around the expandable portion, instead of parallel to the longitudinal axis.

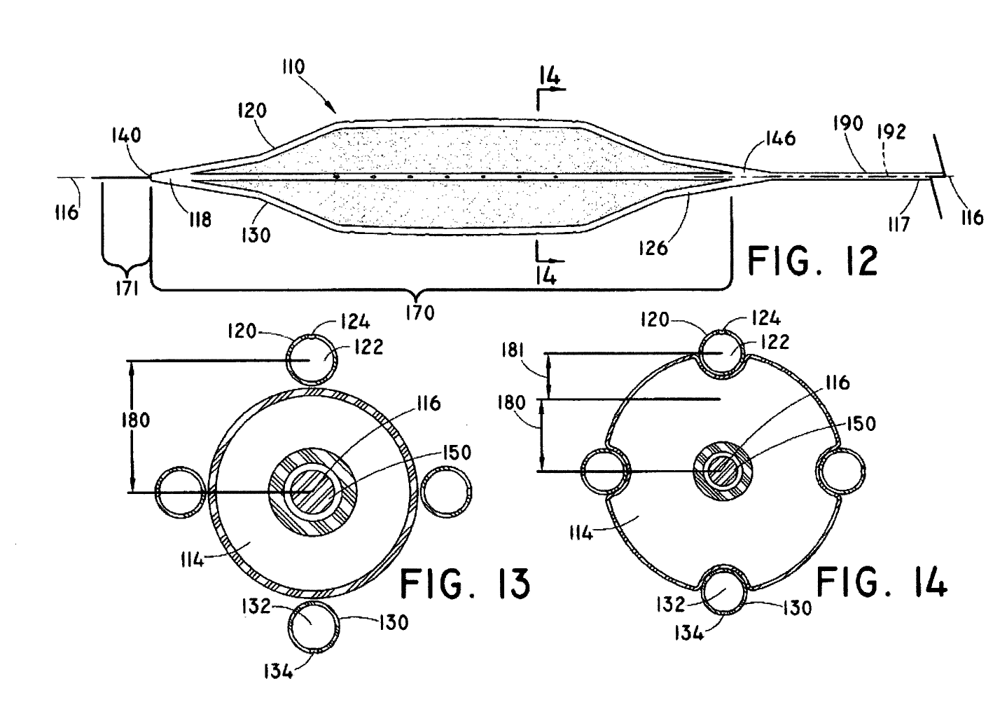

FIGS. 11-15 show a second therapeutic agent delivery device 110, without a balloon catheter. Optionally, the second therapeutic agent delivery device 110 may be configured for use with a balloon catheter having one or more different sizes as part of a kit. FIG. 11 is a perspective view of the second therapeutic agent delivery device 110 in the low-profile configuration and FIG. 12 is a perspective view of the second therapeutic agent delivery device 110 in the expanded configuration, when expanded by an expandable portion 114 (optionally supplied) of a catheter (not shown) along a guidewire 150 (optionally supplied). Alternatively, the second therapeutic agent delivery device 110 may be moved from the low-profile configuration in FIG. 11 to the expanded configuration in FIG. 12 by translating the distal tip 40 in the proximal direction along the guide wire 250. For example, a restraining means such as a tether or wire may be attached to the distal tip 40 and extend through the catheter shaft. By pulling on the restraining means, a user may longitudinally translate the distal tip 40 toward the proximal base 146, bowing the conduits 20 radially outward from the longitudinal axis to assume the expanded configuration shown in FIG. 12.

Referring to FIG. 11 and FIG. 12, a preferred embodiment of a therapeutic agent delivery device 110 comprises a first therapeutic agent delivery conduit 120, a second therapeutic agent delivery conduit 130, or more therapeutic agent delivery conduits, a distal tip 140, and a proximal base 146, which are substantially similar to the corresponding portions of the first therapeutic agent delivery device 10 described above. The distal tip 140 joins the first and second therapeutic agent delivery conduits 120, 130 and includes an annular opening 142 aligned along a longitudinal axis 116. The proximal base 146 is separated proximally from the distal tip 140 by a longitudinal distance 170 and joins the first and second therapeutic agent delivery conduits 120, 130. The proximal base 146 is also adaptable for receiving a catheter shaft (not shown) with the expandable portion 114 (optionally supplied) along a guide wire 150 (optionally supplied). The first and second therapeutic agent delivery conduits 120, 130 are separated from each other and moveable independent of each other between the distal tip 140 and the proximal base 146.

FIG. 13 is a cross sectional view taken along line 13-13 of the therapeutic agent delivery device in FIG. 11. As shown in FIG. 13, each therapeutic agent delivery conduit 120, 130 is positioned at a radial distance 180 from the longitudinal axis 116 when each therapeutic agent delivery conduit 120, 130 is in a low-profile configuration and the longitudinal distance 170 of the distal tip 140 and the proximal base 146 is at a maximum distance (FIG. 11). FIG. 14 is a cross sectional view taken along line 14-14 of the therapeutic agent delivery device in FIG. 12. As shown in FIG. 14, each therapeutic agent delivery conduit 120, 130 resiliently bends from the low-profile configuration (FIG. 11) to an expanded configuration (FIG. 12) upon longitudinal translation of the distal tip 140 toward the proximal base 146 along the longitudinal axis 116. A portion 126 of the therapeutic agent delivery conduit is shown to bend resiliently in FIG. 12. When the distal tip 140 translates toward the proximal base 146 from a maximal longitudinal distance 170 to a smaller distance by proximal translation along a longitudinal distance 171, the radial distance 180 from the longitudinal axis 116 of each therapeutic agent delivery conduit increases to larger radial distance by an increased radial distance 181. When this occurs, each therapeutic agent delivery conduit 120, 130 bends from the low-profile configuration (FIG. 11) to the expanded configuration (FIG. 12).

FIG. 11 and FIG. 12 further depict the first therapeutic agent delivery conduit 120 having a first therapeutic agent delivery lumen 122 and a first therapeutic agent delivery port 124. The first therapeutic agent delivery port 124 is in communication with the first therapeutic agent delivery lumen 122. The first therapeutic agent delivery port 124 faces away from the second therapeutic agent delivery conduit 130. The second therapeutic agent delivery conduit 130 includes a second therapeutic agent delivery lumen 132 and a second therapeutic agent delivery port 134. The second therapeutic agent delivery port 134 is in communication with the second therapeutic agent delivery lumen 132. The second therapeutic agent delivery port 134 faces away from the first therapeutic agent delivery conduit 120.

Preferably, one or more of the therapeutic agent delivery conduits 120, 130 can include a plurality of therapeutic agent delivery ports 124, 134. The ports 124, 134 may be disposed or sized similarly to ports 24, 34 as discussed previously. The ports 124, 134 can have different cross sectional areas. For example, the plurality of ports 124 may include a distal therapeutic agent delivery port 124a located distally to a proximal therapeutic agent delivery port 124b. The distal therapeutic agent delivery port 124a may have a larger cross-sectional area than the proximal therapeutic agent delivery port 124b. Similarly, the plurality of ports 134 may include a distal therapeutic agent delivery port 134a located distally to proximal therapeutic agent delivery port 134b. The distal therapeutic agent delivery port 134a may have a larger cross-sectional area than the proximal therapeutic agent deliver port 134b. The plurality of ports 134 may be similarly sized and situated as the plurality of ports 124, or may be differently sized or situated. Further with respect to accommodating fluid pressure losses and providing even distribution, the lumens 122, 132 may also have portions of different cross-sectional areas. For example, the cross-sectional area of the lumens 122, 132 may decrease or taper distally along the lumen from the proximal base 146 or proximal end 117. Alternatively, the cross-sectional area of the lumens 122, 132 may increase or enlarge distally along the lumen from the proximal base 146 or proximal end 117.

The second therapeutic agent delivery device 110 may further comprise a drug delivery conduit 190 extending in the proximal direction from the proximal end 117. The drug delivery conduit 190 defines a third therapeutic agent delivery lumen 192 that is in communication with the first therapeutic agent delivery lumen 122. The third therapeutic agent delivery lumen 192 also can be in further communication with the second therapeutic agent delivery lumen 132.

As shown in FIG. 11 and FIG. 12, the second therapeutic agent delivery device 110 is adapted to receive one or more differently-sized catheter shafts. The catheter shaft may be placed between the conduits 120, 130 with an expandable catheter portion placed between the therapeutic agent delivery conduits 120, 130 and the distal tip 140 securable within the distal tip 140. In one aspect, kits comprising one or more catheters comprising an expandable portion may be combined with a therapeutic agent delivery device 110. The kit can also comprise a catheter shaft comprising an expandable portion similar to the therapeutic agent delivery system 10 discussed previously. The expandable portion of a catheter may be positioned between two or more of the conduits 120, 130 and between the proximal base 146 and the distal tip 140, where the expandable portion may be inflated from a deflated configuration to an inflated configuration within a body vessel. The catheter shaft may extend from a proximal end 117 of the drug delivery conduit 190 along the longitudinal axis 116 to a distal end 118 positioned between the distal tip 140 and the expandable portion. The catheter shaft also may contact the proximal base 146 between the expandable portion and the proximal end 117. The catheter shaft also includes an inflation lumen in communication with the expandable portion and extends from the proximal end 117 to the expandable portion.

In a second embodiment, methods of delivering a therapeutic agent to a body vessel are provided. The methods include the step of inserting a therapeutic delivery system, such as the systems described with respect to the first embodiment above, within a body vessel in a low-profile (radially compressed) configuration. The therapeutic agent delivery system preferably includes at least one conduit moveable from the low-profile configuration to an expanded configuration within a body vessel. The conduit contains one or more ports configured and adapted to release a therapeutic agent. In the expanded configuration, the conduit is adapted to release the therapeutic agent through one or more ports into direct contact with the wall of a body vessel, preferably without cutting or scoring the wall of the body vessel. The conduit may be pressed into the body vessel in the expanded configuration so as to shape the body vessel around the conduit, causing the body vessel to continuously wrap around a portion of the conduit having a port. The therapeutic agent may be expelled through the port at a pressure sufficient to further distend the wall of the body vessel, creating a sinus region containing the therapeutic agent. During and after ejecting the therapeutic agent through the port, the conduit may be maintained in the expanded configuration for a period of time effective to permit absorption of the therapeutic agent into portions of the body vessel contacting the conduit or forming the sinus region surrounding the ejected therapeutic agent. The therapeutic agent may be ejected from the port with a pressure adequate to distend a portion of the body vessel wall contacting or wrapping around the conduit, forming a sinus containing the therapeutic agent trapped between the body vessel wall and the conduit, without scoring or cutting the wall of the body vessel. The conduit(s) of the medical device may be moved from the low-profile configuration to the expanded configuration my any suitable means, such as expansion of an expandable member (e.g., a catheter balloon) enclosed by one or more conduit(s), or a means for longitudinally translating a distal tip toward the proximal end, therby bowing the conduit arm(s) radially outward into the expanded configuration.

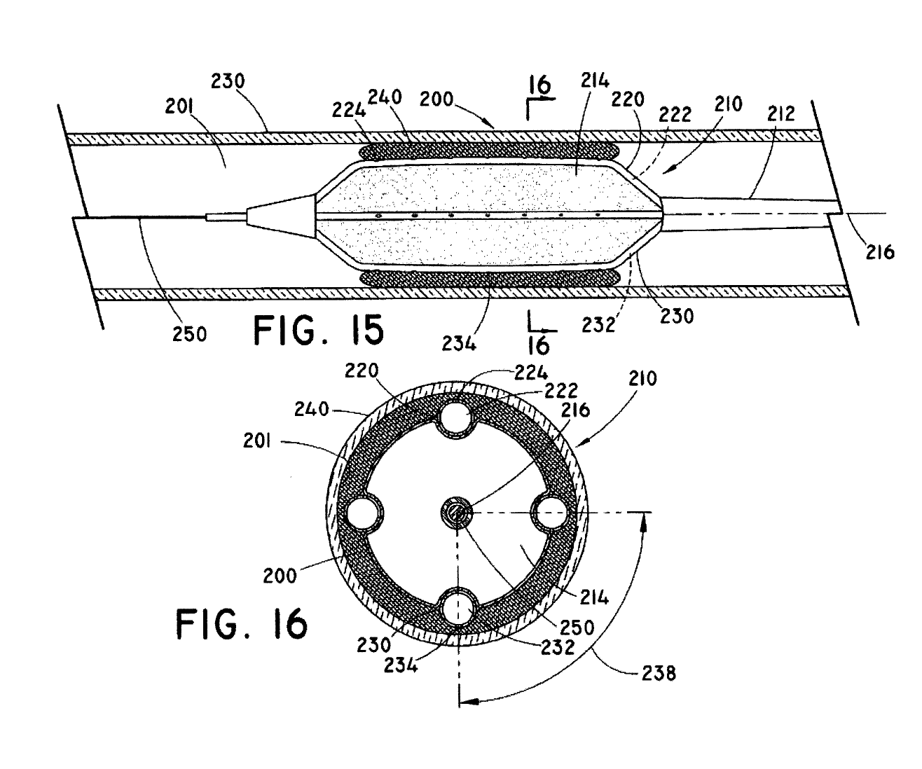

In one aspect of the second embodiment, the therapeutic agent is delivered to an interior wall 201 of a body vessel 230 at or near a treatment site 200 are provided, as shown in FIG. 15 and FIG. 16. FIG. 16 is a cross sectional view taken along line 16-16 of the second therapeutic agent delivery device 210 in FIG. 15. The therapeutic agent delivery system 210 may be configured as described with respect to the first embodiment (10, 110), preferably comprising a catheter shaft and at least one therapeutic agent delivery conduit 220. Optionally, the catheter shaft may include an expandable portion 214 that is inflatable from a deflated configuration to an inflated configuration. Furthermore, the catheter shaft extends along a longitudinal axis 216 from a proximal end to a distal end and includes an inflation lumen in communication with the expandable portion 214. The therapeutic agent delivery conduit 220 includes a therapeutic agent delivery lumen 222 and also includes a therapeutic agent delivery port 224 in communication with the therapeutic agent delivery lumen 222. The therapeutic agent delivery conduit 220 is positioned external to the expandable portion 214 of the catheter shaft 212 and contacts at least a portion of an external surface of the expandable portion 214 while the expandable portion 214 is in the inflated configuration. The therapeutic agent delivery conduit 220 preferably moves independently of the expandable portion 214 while the expandable portion 214 is in the deflated configuration.

The therapeutic agent delivery system 210 may be inserted into a body vessel by any suitable technique. Typically, the therapeutic agent delivery system 210 is inserted by being pushed along a guide wire 250 already inserted into the body vessel. A portion of the at least one therapeutic agent delivery conduit 220, which contacts the external surface of the expandable portion 214 in the inflated configuration and has the therapeutic agent delivery port 224, is positioned proximate the treatment site 200. The treatment site 200 is typically within an artery or vein, preferably a peripheral vascular site in the arms or legs. Examples of suitable peripheral arterial vascular sites include: iliac arteries, femoropopliteal arteries, infrapopliteal arteries, femoral arteries, superficial femoral arteries, popliteal arteries, and the like. Alternatively, the treatment site 200 is present in a heart associated vessel, e.g. the aorta, a coronary artery or branch vessel thereof, or a carotid artery or a branch vessel thereof. In one example, the present invention can be used in contralateral superficial femoral artery (SFA) vessel advancement for critical limb salvage cases, which may be particularly useful in treating diabetic patients. Similarly, the present invention can be used to affect various procedures in the abdominal or femoral arteries, and can be used to treat occlusive peripheral vascular disease, critical limb ischemia, and other related conditions. The medical devices described with respect to the first embodiment may be placed in a body vessel to treat peripheral vascular disease, for example by releasing a therapeutic agent within a peripheral blood vessel. Peripheral vascular disease (PVD) is a common condition with variable morbidity affecting mostly men and women older than 50 years. Peripheral vascular disease of the lower extremities comprise a clinical spectrum that goes from asymptomatic patients, to patients with chronic critical limb ischemia (CLI) that might result in amputation and limb loss. Methods of treating peripheral vascular disease, including critical limb ischemia, preferably comprise the endovascular implantation of one or more coated medical devices provided herein. Atherosclerosis underlies many cases of peripheral vascular disease, as narrowed vessels that cannot supply sufficient blood flow to exercising leg muscles may cause claudication, which is brought on by exercise and relieved by rest. As vessel narrowing increases, critical limb ischemia (CLI) can develop when the blood flow does not meet the metabolic demands of tissue at rest. While critical limb ischemia may be due to an acute condition such as an embolus or thrombosis, most cases are the progressive result of a chronic condition, most commonly atherosclerosis. The development of chronic critical limb ischemia usually requires multiple sites of arterial obstruction that severely reduce blood flow to the tissues. Critical tissue ischemia can be manifested clinically as rest pain, nonhealing wounds (because of the increased metabolic requirements of wound healing) or tissue necrosis (gangrene).

Once placed at the treatment site 200, the expandable portion 214 of the catheter shaft is inflated. The inflation may be performed in a therapeutically effective manner. For example, the inflation may be performed gradually for about 1 minute to 10 minutes to about 30 minutes in stepped increments until at least the portion of the external surface of the expandable portion 214 contacts the at least one therapeutic agent delivery conduit 220, as shown in FIG. 16. Then the pressure of the expandable portion 214 of the catheter shaft may be increased until the at least one therapeutic agent delivery conduit 220 is pressed into a portion 240 of the wall 201 of the body vessel as shown in FIG. 15 and FIG. 16. By pressing the at least one conduit 220 into the body vessel wall 201, not only does the expandable portion 214 of the catheter shaft seal and prevent blood flow, but also a seal is created in between the at least one conduit 220 and the body vessel wall 201. A therapeutic agent can then be injected into the therapeutic agent delivery lumen 222 to release the therapeutic agent through the therapeutic agent delivery port 224 to the wall 201 of the body vessel proximate the treatment site 200. The seal between the at least one conduit 220 and the body vessel wall 201 directs the therapeutic agent into the body vessel wall 201. In addition, with the pressing of the at least one conduit 220 into the body vessel wall 201, sufficient penetration of the body vessel wall 201 is achieved, thereby allowing the effective administration of the therapeutic agent into the body vessel. The at least one therapeutic agent delivery conduit 220 also provides areas of increased pressure to the treatment site 200. Inducing higher stress upon the treatment site 200 would help disrupt plaque buildup. Preferably, these areas of increased pressure would not be “sharp” enough to perforate the body vessel wall 201 or cause undesired harm. After treatment, the expandable portion 214 of the catheter shaft is deflated and the therapeutic agent delivery system 210 is removed from the body vessel.

Alternatively, the method of delivering one or more therapeutic agents can be delivered with a therapeutic agent delivery system 210 with at least two therapeutic agent delivery conduits 220. Referring to FIGS. 15 and 16, the therapeutic agent delivery system 210 can include at least one at least one first therapeutic agent delivery conduit 220 including a first therapeutic agent delivery lumen 222 and a port 224 in communication with the first therapeutic agent delivery lumen 222. The at least one first therapeutic agent delivery conduit 220 can be positioned external to the expandable portion 214 of the catheter shaft and moveable independent of the expandable portion 214 in the deflated configuration. The port 224 of the at least one first therapeutic agent delivery conduit 220 can face away from a first portion of an external surface of the expandable portion 214 of the catheter shaft for contacting the at least one first therapeutic agent delivery conduit 220 when the expandable portion 214 is in the inflated configuration. Moreover, the therapeutic agent delivery system 210 can also include at least one second therapeutic agent delivery conduit 220 including a second therapeutic agent delivery lumen 232 and a port 234 in communication with the second therapeutic agent delivery lumen 232. The at least one second therapeutic agent delivery conduit 220 can be positioned external to the expandable portion 214 of the catheter shaft and moveable independent of the expandable portion 214 in the deflated configuration. The port of the at least one second therapeutic agent delivery conduit 220 can face away from a second portion of the external surface of the expandable portion of the catheter shaft for contacting the at least one second therapeutic agent delivery conduit when the expandable portion is in the inflated configuration.

Once placed at the treatment site 200, the expandable portion 214 of the catheter shaft may be inflated for about 1 minute to about 30 minutes in stepped increments until at least the portion of the external surface of the expandable portion 214 contacts the at least one of the first and second therapeutic agent delivery conduits 220, as shown in FIG. 16. Other suitable inflation times include 5, 10, 15, and 25 minutes. Then the pressure of the expandable portion 214 of the catheter shaft may be increased until the at least one first and second therapeutic agent delivery conduits 220 are pressed into a portion 240 of the wall 201 of the body vessel as shown in FIG. 15 and FIG. 16. By pressing the at least one first and second conduits 220 into the body vessel wall 201, not only does the expandable portion 214 of the catheter shaft seal and prevent blood flow, but a seal may also be created between the at least one first and second conduits 220 and the body vessel wall 201. A therapeutic agent can then be injected into the at least one therapeutic agent delivery lumens 222, 232 to release the therapeutic agent through the therapeutic agent delivery port 224, port 234, or both to the wall 201 of the body vessel proximate the treatment site 200. The seal between the at least one first and second conduits 220 and the body vessel wall 201 directs the therapeutic agent into the body vessel wall 201. A therapeutically effective rate of ejection of the therapeutic agent through the ports 224, 234 may be selected. For instance, ejection of the therapeutic agent may distend the wall of the body vessel radially outward away from the ports 224, 234, forming a sinus region that retains the therapeutic agent between the conduit 220 and the portion of the body vessel wall 201 wrapped around the conduit 220. By pressing the at least one first and second conduits 220 into the body vessel wall 201, sufficient penetration of the body vessel wall 201 is achieved to allow the effective administration of the therapeutic agent into the body vessel without scoring or cutting the body vessel wall 201. The at least one first and second therapeutic agent delivery conduits 220 also provide areas of increased pressure to the treatment site 200. Inducing higher stress upon the treatment site 200 would help disrupt plaque buildup. Preferably, these areas of increased pressure would not be “sharp” enough to perforate the body vessel wall 201 or cause undesired harm. After treatment, the expandable portion 214 of the catheter shaft is deflated and the therapeutic agent delivery system is removed from the body vessel.

A second therapeutic agent, or more, can also be injected into the second therapeutic delivery lumen 232 of the at least one second therapeutic agent delivery conduit 220. The second therapeutic agent can be released through the port 234 to the body vessel wall 201 proximate the treatment site. Preferably, the first and second therapeutic agent delivery lumens 222, 232 are isolated from one another where it is desirable to introduce at least two therapeutic agents to the treatment site simultaneously, or shortly thereafter. In one embodiment, the at least one first and second therapeutic agent delivery conduits 220 circumferentially alternate about the expandable portion when inflated. This embodiment will allow a more effective and equal distribution of the first and second therapeutic agents throughout the body vessel wall 201. Alternatively, the at least one first and second therapeutic agent delivery conduits 220 may not circumferentially alternate about the expandable portion 214 when inflated, but instead may be grouped. In this embodiment, it may be more desirable to deliver two therapeutic agents in isolation to two different regions of the body vessel wall 201. The at least one first and second therapeutic agent delivery conduits 220 may also be circumferentially spaced apart from one another by a circumferential distance 238 measured perpendicular to the longitudinal axis 216 when the expandable portion 214 is in the inflated configuration. Preferably, the circumferential distances 238 are substantially equal.

These methods of locally administering therapeutic agents with the therapeutic agent delivery system 210 described herein could eliminate the need of cutting balloons with cutting balloon angioplasty, and eliminate the additional steps of providing an infusion catheter delivering therapeutic agents. The methods of treatment may be performed without one or more of the following steps: (i) inserting the cutting balloon and manipulating the cutting balloon to score the body vessel wall to accommodate the release of the therapeutic agent beneath the surface of the body vessel wall or (ii) inserting the infusion catheter to deliver a therapeutic agent to the body vessel wall, and preferably, to scored regions underneath the body vessel wall. In contrast, the preferred methods of treatment can be performed without requiring these steps. Instead of scoring, the method of increasing pressure to the at least one of the first and second therapeutic agent delivery conduits 220 can provide areas of increased pressure to the treatment site 200. Inducing higher stress upon the treatment site 200 would help disrupt plaque buildup. Preferably, these areas of increased pressure would not be “sharp” enough to perforate the body vessel wall 201 or cause undesired harm or trauma, unlike scoring with cutting balloons. Furthermore, during the inflation of the expandable portion 214, therapeutic agents can be introduced to perform multiple functions including modulating angiogenesis, restenosis, cell proliferation, thrombosis, platelet aggregation, clotting, and vasodilation to prepare the region for the penetration of conduits. Additionally, after suitable inflation, subsequent therapeutic agents can be delivered to perform multiple functions including modulating angiogenesis, restenosis, cell proliferation, thrombosis, platelet aggregation, clotting, and vasodilation during the engagement of the therapeutic agent delivery system 210 or to perform such functions after the removal of the therapeutic agent delivery system 210.

The following are particularly preferred methods. In one example, a method of delivering a therapeutic agent to an interior wall of a body vessel at or near a treatment site, the method comprising the steps of:

(a) inserting a therapeutic agent delivery system into a body vessel, the therapeutic agent delivery system comprising:
(i) a catheter shaft having an expandable portion being inflatable between a deflated configuration and an inflated configuration, the catheter shaft extending along a longitudinal axis from a proximal end to a distal end and including an inflation lumen in communication with the expandable portion, and
(ii) a therapeutic agent delivery conduit including a therapeutic agent delivery lumen and a port in communication with the therapeutic agent delivery lumen; the therapeutic agent delivery conduit positioned external to the expandable portion of the catheter shaft and moveable independent of the expandable portion in the deflated configuration;

(b) positioning a portion of the therapeutic agent delivery conduit having the port proximate the treatment site;

(c) inflating the expandable portion of the catheter shaft until at least a portion of the external surface of the expandable portion contacts the therapeutic agent delivery conduit; and

(d) injecting a therapeutic agent into the therapeutic agent delivery lumen of the therapeutic agent delivery conduit to release the therapeutic agent through the port to a wall of the body vessel proximate the treatment site.

This method may further comprise one or more of the following steps: (1) the step of increasing the pressure of the expandable portion of the catheter shaft until the therapeutic agent delivery conduit is pressed into the body vessel wall and the port is sealably engaged with the body vessel wall; and/or (2) deflating the expandable portion of the catheter shaft, and removing the therapeutic agent delivery system from the body vessel.

Optionally, the therapeutic agent delivery system further comprises a plurality of therapeutic agent delivery conduits each including a therapeutic agent delivery lumen and a port in communication with said therapeutic agent delivery lumen; each therapeutic agent delivery conduit positioned external to the expandable portion of the catheter shaft and moveable independent of the expandable portion in the deflated configuration. A portion of each therapeutic agent delivery lumen of the plurality of therapeutic agent delivery conduits may be in fluid communication. The therapeutic agent delivery conduit may further comprise a plurality of ports in communication with the therapeutic agent delivery lumen and facing away from the portion of the external surface of the expandable portion for contacting the therapeutic agent delivery conduit. The plurality of ports may be disposed longitudinally along the therapeutic agent delivery conduit in a substantially straight line. The plurality of ports may include a first port located distally to a second port, the first port having a larger cross-sectional area than the second port. The catheter shaft may have a proximal portion extending from a distal end of the expandable portion to the proximal end of the catheter shaft, and where the therapeutic agent delivery conduit and the catheter shaft may be coaxially oriented about the longitudinal axis at said proximal portion. In addition, the catheter shaft may have a proximal portion extending from a distal end of the expandable portion to the proximal end of the catheter shaft, said proximal portion including a portion of the therapeutic agent delivery lumen proximal to, and in communication with, the therapeutic agent delivery conduit.

In another example, a method of delivering a therapeutic agent to an interior wall of a body vessel at or near a treatment site, the method comprising the steps of:

(a) inserting a therapeutic agent delivery system into a body vessel, the therapeutic agent delivery system comprising:
(i) a catheter shaft having an expandable portion being inflatable between a deflated configuration and an inflated configuration, the catheter shaft extending along a longitudinal axis from a proximal end to a distal end and including an inflation lumen in communication with the expandable portion,
(ii) at least one first therapeutic agent delivery conduit including a first therapeutic agent delivery lumen and a port in communication with the first therapeutic agent delivery lumen; the at least one first therapeutic agent delivery conduit positioned external to the expandable portion of the catheter shaft and moveable independent of the expandable portion in the deflated configuration, the port of the at least one first therapeutic agent delivery conduit facing away from a first portion of an external surface of the expandable portion of the catheter shaft for contacting the at least one first therapeutic agent delivery conduit when the expandable portion is in the inflated configuration, and
(iii) at least one second therapeutic agent delivery conduit including a second therapeutic agent delivery lumen and a port in communication with the second therapeutic agent delivery lumen; the at least one second therapeutic agent delivery conduit positioned external to the expandable portion of the catheter shaft and moveable independent of the expandable portion in the deflated configuration, the port of the at least one second therapeutic agent delivery conduit facing away from a second portion of the external surface of the expandable portion of the catheter shaft for contacting the at least one second therapeutic agent delivery conduit when the expandable portion is in the inflated configuration;

(b) positioning a portion of the at least one first and second therapeutic agent delivery conduits having the port proximate the treatment site;

(c) inflating the expandable portion of the catheter shaft until the first and second portions of the external surface of the expandable portion contact at least one first therapeutic agent delivery conduit and at least one second therapeutic agent delivery conduit, respectively; and

(d) injecting a first therapeutic agent into at least one of the first and second therapeutic agent delivery lumens to release the first therapeutic agent through at least one of the ports of the at least one first and second therapeutic agent delivery conduits to a wall of the body vessel proximate the treatment site.

The method may further comprise the step of injecting a second therapeutic agent into the second therapeutic agent delivery lumen of the at least one second therapeutic agent delivery conduit to release the second therapeutic agent through the port of the at least one second therapeutic agent delivery conduit to the body vessel wall proximate the treatment site. The method may also further comprise the step of increasing the pressure of the expandable portion of the catheter shaft until the at least one first and second therapeutic agent delivery conduits are pressed into the body vessel wall and each port is sealably engaged with the body vessel wall. In addition, or in the alternative, the method may further comprise the steps of deflating the expandable portion of the catheter shaft, and removing the therapeutic agent delivery system from the body vessel.

Optionally, a distal end of the at least one first and second therapeutic agent delivery conduits may be joined to one another to form a distal tip positioned distally to the expandable portion of the catheter shaft and moveable independent of the expandable portion in the deflated configuration, the distal tip including an annular opening adapted for receiving a guide wire. The at least one first therapeutic agent delivery conduit may further comprise a plurality of ports in communication with the first therapeutic agent delivery lumen and facing away from the first portion of the external surface of the expandable portion, and the at least one second therapeutic agent delivery conduit may further comprise a plurality of ports in communication with the second therapeutic agent delivery lumen and facing away from the second portion of the external surface of the expandable portion. The plurality of ports may be disposed longitudinally along each of the at least one first and second therapeutic agent delivery conduits in a substantially straight line, the plurality of ports of each of the at least one first and second therapeutic agent delivery conduits including a first port located distally to a second port, the first port having a larger cross-sectional area than the second port. In addition or in the alternative. The at least one first therapeutic agent delivery conduit and the at least one second therapeutic agent delivery conduit may be disposed circumferentially and/or may be spaced apart from one another by a circumferential distance measured perpendicular to the longitudinal axis, the circumferential distance between each of the at least one first and second therapeutic agent delivery conduits being substantially equal. The catheter shaft may have a proximal portion extending from a distal end of the expandable portion to the proximal end of the catheter shaft, said proximal portion including a portion of the first therapeutic agent delivery lumen proximal to, and in communication with, the at least one first therapeutic agent delivery conduit, and a portion of the second therapeutic agent delivery lumen proximal to, and in communication with, the at least one second therapeutic agent delivery conduit.

This method of locally administering therapeutic agents could also eliminate the need for a stent or at least the need for a stent to deliver the therapeutic agent. In a first aspect, the therapeutic agents may alter the composition of the stenosis such that the stenosis breaks down. The method of the invention can be used to treat disorders by delivery of any composition, e.g., drug or gene with a catheter, as described herein. For example, patients with peripheral arterial disease, e.g., critical limb ischemia (Isner, J. M. et al, Restenosis Summit VIII, Cleveland, Ohio, 1996, pp 208-289) can be treated as described herein. Any composition that inhibits smooth muscle cell (SMC) proliferation and migration, platelet aggregation and extracellular modeling is also desirable.

In a first aspect, the therapeutic agent may be, for example, any bioactive material selected for a desired therapeutic effect. In particular, therapeutic agents preferably inhibit or mitigate one or more events implicated in the restenosis process, such as: (a) destruction of endothelial and subendothelial structures, (b) traumatization of medial regions with rupture of the internal elastic lamina, (c) release of thrombogenic factors such as collagen or tissue factor, (d) stretching of smooth muscle cells with subsequent expression of proto-oncogenes (c-fos, c-myc, c-myb), (e) release of growth factors from cells of the bloodstream, endothelial cells and SMCs, and (f) thrombin production with autocatalytic activation of the SMC thrombin receptor. Overlapping the inflammation period, granulation begins 3 days after angioplasty. Proteinases such as plasmin as well as collagenases induce the disintegration of extracellular matrix structures, thereby modulating plaque formation, and lead to an organelle-rich SMC phenotype within the intima and media. Overlapping with the granulation period, induction of different components of the extracellular matrix occurs 12 weeks after angioplasty, possibly mediated by TGF-beta (phase of matrix formation). Smooth muscle cells produce and secrete matrix proteins such as tenascin, fibronectin, collagens and proteoglycans, and thereby induce a marked increase of the neointimal plaque volume.

For example, the therapeutic agent may be an antisense compound is selected to interact within a cell to inhibit or mitigate restenosis by inhibiting the activity of mRNA produced from proto-oncogenes such as c-myc. C-myc is a proto-oncogene which regulates cell growth and differentiation, is involved in the process of vascular remodeling, regulating smooth muscle cell proliferation and extracellular matrix synthesis, in addition to playing a role in apoptosis. As used herein, the term “antisense” refers to a molecule that binds to a messenger RNA (mRNA) or a nucleic acid molecule that hybridizes to such a molecule. For example, the antisense compound may be an oligomer having a particular sequence of nucleotide bases and a subunit-to-subunit backbone that allows the antisense oligomer to form an RNA:oligomer heteroduplex within the target sequence, typically with an mRNA. The oligomer may have exact sequence complementarity to the target sequence or near complementarity. These antisense oligomers may block or inhibit translation of the mRNA, and/or modify the processing of an mRNA to produce a splice variant of the mRNA. Preferred antisense compounds are those that interact with the c-myc gene, for example by binding to mRNA produced by the gene. The therapeutic agent may be a c-myc antisense compound, preferably a nuclease-resistant antisense morpholino compound having high affinity (i.e., “specifically hybridizes”) to a complementary or near-complementary c-myc nucleic acid sequence, e.g., the sequence including and spanning the normal AUG start site. Preferred c-myc antisense compounds are described in U.S. Pat. No. 7,094,765 to Iversen et al., filed Jan. 29, 2000, the portion of which pertaining to the synthesis, sequences and administration of c-myc antisense compounds is incorporated herein by reference. Preferably, the antisense compounds include a morpholino backbone structure. In particular, the therapeutic agent may be a morpholino antisense compound having (i) a polynucleotide (preferably containing from 8 to 40 nucleotides) including a targeting base sequence that is complementary to a region that spans the translational start codon of a c-myc mRNA and (ii) uncharged, phosphorous-containing intersubunit linkages. The synthesis, structures, and binding characteristics of such morpholino oligomers are detailed in above-cited U.S. Pat. Nos. 5,698,685, 5,217,866, 5,142,047, 5,034,506, 5,166,315, 5,521,063, and 5,506,337, all of which are incorporated herein by reference. The antisense oligomers therapeutic agents are preferably composed of morpholino subunits of the form shown in the above cited patents, where (i) the morpholino groups are linked together by uncharged phosphorus-containing linkages, one to three atoms long, joining the morpholino nitrogen of one subunit to the 5′ exocyclic carbon of an adjacent subunit, and (ii) the base attached to the morpholino group is a purine or pyrimidine base-pairing moiety effective to bind, by base-specific hydrogen bonding, to a base in a polynucleotide. The purine or pyrimidine base-pairing moiety is typically adenine, cytosine, guanine, uracil or thymine. Preparation of such oligomers is described in detail in U.S. Pat. No. 5,185,444 (Summerton and Weller, 1993), which is hereby incorporated by reference in its entirety. As shown in the reference, several types of nonionic linkages may be used to construct a morpholino backbone.

Other examples of suitable therapeutic agents include antiproliferative agents, an antineoplastic agent, an antibiotic agent, an anti-inflammatory agent and/or a free radical scavenger. Therapeutic agents may perform multiple functions including modulating angiogenesis, restenosis, cell proliferation, thrombosis, platelet aggregation, clotting, and vasodilation. More specifically, the therapeutic agent may be paclitaxel, dexamethasone, rapamycin (sirolimus), a rapamycin analog (including tacrolimus or everolimus), a nonsteroidal anti-inflammatory drug and/or a steroidal anti-inflammatory drug. The therapeutic agent may also include a pH-altering substance, such as an acid or base, selected to dissolve a vascular blockage. For treatment of vascular calcified occlusions with the therapeutic agent delivery systems, an acidic dissolution fluid may be delivered for a period of time sufficient for fluid flow to be to be enhanced through the vascular site, for example as described by Delaney in U.S. Pat. No. 6,290,689, filed Oct. 22, 1999.

In another aspect, the therapeutic agent delivery system could be used to treat post-deep vein thrombosis (DVT) patients. The therapeutic agent delivery system could be used to deliver thrombolytics agents to the body vessel wall 201, which creates a chain reaction of thrombis breakdown. After breakdown, further dilation of the therapeutic agent delivery system would restore the vessel to its native diameter. Examples of suitable thrombolytic therapeutic agents include anticoagulant agents, antiplatelet agents, antithrombogenic agents and fibrinolytic agents. Anticoagulants are bioactive materials which act on any of the factors, cofactors, activated factors, or activated cofactors in the biochemical cascade and inhibit the synthesis of fibrin. Antiplatelet bioactive materials inhibit the adhesion, activation, and aggregation of platelets, which are key components of thrombi and play an important role in thrombosis. Fibrinolytic bioactive materials enhance the fibrinolytic cascade or otherwise aid is dissolution of a thrombus. Examples of antithrombotics include but are not limited to anticoagulants such as thrombin, Factor Xa, Factor VIIa and tissue factor inhibitors; antiplatelets such as glycoprotein IIb/IIIa, thromboxane A2, ADP-induced glycoprotein IIb/IIIa, and phosphodiesterase inhibitors; and fibrinolytics such as plasminogen activators, thrombin activatable fibrinolysis inhibitor (TAFI) inhibitors, and other enzymes which cleave fibrin. Further examples of antithrombotic bioactive materials include anticoagulants such as heparin, low molecular weight heparin, covalent heparin, synthetic heparin salts, coumadin, bivalirudin (hirulog), hirudin, argatroban, ximelagatran, dabigatran, dabigatran etexilate, D-phenalanyl-L-poly-L-arginyl, chloromethy ketone, dalteparin, enoxaparin, nadroparin, danaparoid, vapiprost, dextran, dipyridamole, omega-3 fatty acids, vitronectin receptor antagonists, DX-9065a, CI-1083, JTV-803, razaxaban, BAY 59-7939, and LY-51,7717; antiplatelets such as eftibatide, tirofiban, orbofiban, lotrafiban, abciximab, aspirin, ticlopidine, clopidogrel, cilostazol, dipyradimole, nitric oxide sources such as sodium nitroprussiate, nitroglycerin, S-nitroso and N-nitroso compounds; fibrinolytics such as alfimeprase, alteplase, anistreplase, reteplase, lanoteplase, monteplase, tenecteplase, urokinase, streptokinase, or phospholipid encapsulated microbubbles; and other bioactive materials such as endothelial progenitor cells or endothelial cells.

The dosage ranges for the administration of the therapeutic agent in the methods of treatment are those large enough to produce the desired effect in which the symptoms of the disease/injury are ameliorated. The dosage should not be so large as to cause adverse side effects. Generally, the dosage will vary with the age, condition, sex and extent of the disease in the patient and can be determined by one of skill in the art. The dosage can be adjusted by the individual physician in the event of any complication. When used for the treatment of inflammation, post-reperfusion injury, microbial/viral infection, or vasculitis, or inhibition of the metastatic spread of tumor cells, for example, the therapeutic composition may be administered at a dosage which can vary from about 1 mg/kg to about 1000 mg/kg, preferably about 1 mg/kg to about 50 mg/kg, in one or more dose administrations.

Instead of administering a therapeutic agent that is effective immediately, it is also possible to embed the therapeutic agent in a bioabsorbable material that allows a controlled release once the material is transferred into the lesion. Optionally, the therapeutic agent may be incorporated into particles of a polymeric substance such as polyesters, polyamino acids, hydrogels, polylactide/glycolide copolymers, or ethylenevinylacetate copolymers. The therapeutic agent may be contained in microcapsules prepared, for example, by coacervation techniques or by interfacial polymerization, for example, by the use of hydroxymethylcellulose or gelatin-microcapsules or poly(methylmethacrolate) microcapsules, respectively, or in a colloid drug delivery system. Colloidal dispersion systems include macromolecule complexes, nanocapsules, microspheres, beads, and lipid-based systems including oil-in-water emulsions, micelles, mixed micelles, and liposomes.

Having described certain preferred embodiments in detail, it will be apparent that modifications and variations are possible without departing from the scope of the invention defined in the appended claims. More specifically, although some aspects of the present invention are identified herein as preferred or particularly advantageous, it is contemplated that the present invention is not necessarily limited to these preferred aspects of the invention.

1. A therapeutic agent delivery system comprising:
a catheter shaft having an expandable portion being inflatable between a deflated configuration and an inflated configuration, the catheter shaft extending along a longitudinal axis from a proximal end to a distal end and including an inflation lumen in communication with the expandable portion; and
a therapeutic agent delivery conduit including a therapeutic agent delivery lumen and a therapeutic agent delivery port in communication with the therapeutic agent delivery lumen; and
the therapeutic agent delivery conduit being positioned external to the expandable portion of the catheter shaft and contacting at least a portion of an external surface of the expandable portion in the inflated configuration, the therapeutic agent delivery conduit being moveable independent of the expandable portion in the deflated configuration.

2. The system of claim 1, wherein at least a portion of the therapeutic agent delivery conduit positioned external to the expandable portion of the catheter shaft is disposed substantially parallel to the longitudinal axis of the catheter shaft when the expandable portion is in the deflated configuration.

3. The system of claim 1, wherein the therapeutic agent delivery conduit further comprises a plurality of therapeutic agent delivery ports facing away from the portion of the external surface of the expandable portion contacting the therapeutic agent delivery conduit.

4. The system of claim 3, wherein the plurality of therapeutic agent delivery ports are disposed longitudinally along the therapeutic agent delivery conduit in a substantially straight line.

5. The system of claim 3, wherein the plurality of therapeutic agent delivery ports includes a first therapeutic agent delivery port located distally to a second therapeutic agent delivery port, the first therapeutic agent delivery port having a larger cross-sectional area than the second therapeutic agent delivery port.

6. The system of claim 1, wherein the expandable portion has a longitudinally curved external surface in the inflated configuration; and
inflation of the expandable portion from the deflated configuration to the inflated configuration bends the therapeutic agent delivery conduit from a substantially straight configuration to a substantially arcuate configuration along the longitudinally curved external surface of the expandable portion in the inflated configuration.

7. The system of claim 1, wherein at least a portion of the therapeutic agent delivery conduit positioned external to the expandable portion of the catheter shaft is disposed substantially parallel to the longitudinal axis of the catheter shaft when the expandable portion is in the deflated configuration,
wherein the portion of the therapeutic agent delivery conduit that is disposed substantially parallel to the longitudinal axis further comprises a plurality of therapeutic agent delivery ports facing away from the portion of the external surface of the expandable portion contacting the therapeutic agent delivery conduit and disposed longitudinally along the portion of the therapeutic agent delivery conduit that is disposed substantially parallel to the longitudinal axis in a substantially straight line.

8. The system of claim 1, wherein the therapeutic agent delivery conduit and the catheter shaft are coaxially oriented along the longitudinal axis from the proximal end of the catheter shaft to the proximal end of the expandable portion of the catheter shaft.

9. The system of claim 1, wherein the catheter shaft has a proximal portion extending from the expandable portion to the proximal end, the proximal portion of the catheter shaft including a portion of the therapeutic agent delivery lumen proximal to, and in communication with, the therapeutic agent delivery conduit.

10. The system of claim 1, wherein said therapeutic agent delivery conduit is a first therapeutic agent delivery conduit and said portion of the external surface of the expandable portion contacting the first therapeutic agent delivery conduit is a first portion of the external surface of the expandable portion; and
wherein the therapeutic agent delivery system further comprises a second therapeutic agent delivery conduit including a second therapeutic agent delivery lumen and a second therapeutic agent delivery port in communication with the second therapeutic agent delivery lumen;
the second therapeutic agent delivery conduit being positioned external to the expandable portion of the catheter shaft and contacting at least a second portion of the external surface of the expandable portion in the inflated configuration, the second therapeutic agent delivery conduit being moveable independent of the expandable portion in the deflated configuration and the first therapeutic agent delivery conduit; and
the second therapeutic agent delivery port facing away from the second portion of the external surface of the expandable portion contacting the second therapeutic agent delivery conduit.

11. The system of claim 10, wherein a distal end of the first therapeutic agent delivery conduit and a distal end of the second therapeutic agent delivery conduit are joined to form a distal tip positioned distally to the expandable portion of the catheter shaft, wherein the distal tip includes an annular opening adapted for receiving a guide wire, the distal tip being moveable independent of the expandable portion in the deflated configuration.

12. The system of claim 11, wherein the first portion of the external surface of the expandable portion and the second portion of the external surface of the expandable portion are circumferentially spaced apart by a circumferential distance measured perpendicular to the longitudinal axis, the circumferential distance being substantially equal.

13. The system of claim 11, wherein each therapeutic agent delivery conduit comprises a plurality of therapeutic agent delivery ports disposed longitudinally along each therapeutic agent delivery conduit in a substantially straight line,
the therapeutic agent delivery ports of each therapeutic agent delivery conduit including a first therapeutic agent delivery port located distally to a second therapeutic agent delivery port, the first therapeutic agent delivery port having a larger cross-sectional area than the second therapeutic agent delivery port,
the first therapeutic agent delivery port facing away from the first portion of the external surface of the expandable portion, and the second therapeutic agent delivery port facing away from the second portion of the external surface of the expandable portion.

14. The system of claim 10, wherein the catheter shaft has a proximal portion extending from the expandable portion to the proximal end, the proximal portion of the catheter shaft including a portion of the first therapeutic agent delivery lumen proximal to, and in communication with, the first therapeutic agent delivery conduit, the proximal portion of the catheter shaft including a portion of the second therapeutic agent delivery lumen proximal to, and in communication with, the second therapeutic agent delivery conduit.

15. A therapeutic agent delivery device comprising:
a first therapeutic agent delivery conduit, a second therapeutic agent delivery conduit, a distal tip joining the first and second therapeutic agent delivery conduits, the distal tip including an annular opening aligned along a longitudinal axis, and a proximal base separated proximally from the distal tip by a longitudinal distance, the proximal base joining the first and second therapeutic agent delivery conduits and being adaptable for receiving a catheter shaft, the first therapeutic agent delivery conduit and the second therapeutic agent delivery conduit being separated from each other, and moveable independent of each other between the distal tip and the proximal base;
the first therapeutic agent delivery conduit including a first therapeutic agent delivery lumen and including a first therapeutic agent delivery port in communication with the first therapeutic agent delivery lumen;
the second therapeutic agent delivery conduit including a second therapeutic agent delivery lumen and including a second therapeutic agent delivery port in communication with the second therapeutic agent delivery lumen;
each therapeutic agent delivery conduit positioned at a radial distance from the longitudinal axis when each therapeutic agent delivery conduit is in a low-profile configuration when the longitudinal distance of the distal tip and the proximal base is at a maximum distance; and
each therapeutic agent delivery conduit resiliently bending between the low-profile configuration and an expanded configuration upon longitudinal translation of the distal tip toward the proximal base along the longitudinal axis, the radial distance from the longitudinal axis of each therapeutic agent delivery conduit increasing upon bending each therapeutic agent delivery conduit from the low-profile configuration to the expanded configuration.

16. The device of claim 15, wherein each therapeutic agent delivery conduit further comprises a plurality of therapeutic agent delivery ports disposed along the therapeutic agent delivery conduit, each of the plurality of therapeutic agent delivery ports including a distal therapeutic agent delivery port located distally to a proximal therapeutic agent delivery port, the distal therapeutic agent delivery port having a larger cross-sectional area than the proximal therapeutic agent delivery port.

17. The device of claim 15, wherein the device further comprises a catheter shaft extending in the proximal direction from the proximal base, the catheter shaft defining a third therapeutic agent delivery lumen in communication with the first therapeutic agent delivery lumen.

18. The device of claim 17, wherein the third therapeutic agent delivery lumen is in further communication with the second therapeutic agent delivery lumen.

19. The device of claim 15, wherein the device further comprises a catheter shaft comprising an expandable portion positioned between the proximal base and the distal tip, the expandable portion being inflatable between a deflated configuration and an inflated configuration, the catheter shaft extending from a proximal end along the longitudinal axis to a distal end positioned between the distal tip and the expandable portion; the catheter shaft contacting the proximal base between the expandable portion and the proximal end; the catheter shaft including an inflation lumen in communication with the expandable portion and extending from the proximal end to the expandable portion.

20. A method of delivering a therapeutic agent to an interior wall of a body vessel at or near a treatment site, the method comprising:
(a) inserting a therapeutic agent delivery system into a body vessel, the therapeutic agent delivery system comprising
(i) a catheter shaft having an expandable portion being inflatable between a deflated configuration and an inflated configuration, the catheter shaft extending along a longitudinal axis from a proximal end to a distal end and including an inflation lumen in communication with the expandable portion; and
(ii) a therapeutic agent delivery conduit including a therapeutic agent delivery lumen and a therapeutic agent delivery port in communication with the therapeutic agent delivery lumen; the therapeutic agent delivery conduit being positioned external to the expandable portion of the catheter shaft and contacting at least a portion of an external surface of the expandable portion in the inflated configuration, and the therapeutic agent delivery conduit being moveable independent of the expandable portion in the deflated configuration;
(b) positioning a portion of the therapeutic agent delivery conduit proximate the treatment site, said therapeutica agent delvery conduit portion contacting the external surface of the expandable portion in the inflated configuration and having the therapeutic agent delivery port;
(c) inflating the expandable portion of the catheter shaft until at least the portion of the external surface of the expandable portion contacts the therapeutic agent delivery conduit;
(d) increasing the pressure of the expandable portion of the catheter shaft until the therapeutic agent delivery conduit is pressed into a wall of the body vessel;
(e) injecting a therapeutic agent into the therapeutic agent delivery lumen of the therapeutic agent delivery conduit to release the therapeutic agent through the therapeutic agent delivery port to the wall of the body vessel proximate the treatment site;
(f) deflating the expandable portion of the catheter shaft; and
(g) removing the therapeutic agent delivery system from the body vessel.
